(19) United States
(12) Patent Application Publication
(10) Pub. No.: US 2007/0249660 A1
(43) Pub. Date: Oct. 25, 2007

(54) Pharmacologically Acceptable Salts of Clopidogrel

(76) Inventors: Beat T. Weber, Zofingen (CH); Michael Karl Levis, Zofingen (CH); Hon Quang Ly, Allschwil (CH)

Correspondence Address: HOFFMANN & BARON, LLP, 6900 JERICHO TURNPIKE, SYOSSET, NY, 11791, US

(21) Appl. No.: 10/590,391

(22) PCT Filed: Feb. 16, 2005
(86) PCT No.: PCT/CH05/00086

§ 371 Date: May 7, 2007

(30) Foreign Application Priority Data
Feb. 24, 2004 (CH) 300/04

(51) Int. Cl. A61K 31/4365; C07D 495/04

(52) U.S. Cl. 514/301; 546/114

(57) ABSTRACT

The invention relates to polymorphous forms of (+)-(S)-clopidogrel hydrogen bromide, described as polymorphous “form A”, polymorphous “form B”, polymorphous “form C”, polymorphous “form D”, polymorphous “form E”, and polymorphous “form F”, in addition to polymorphous forms of (+)-(S)-clopidogrel napsylate, that are described as polymorphous “form A” and polymorphous “form B” and differ in the X-ray powder diffraction diagrams (XRPD) thereof. The invention also relates to the salts clopidogrel besylate, clopidogrel tosylate and clopidogrel oxalate, and to methods for the production thereof.

The present invention refers to salts of Clopidogrel, especially new polymorphic forms of Clopidogrel-hydrobromide, as well as salts of Clopidogrel with benzene sulfonic acid (besylate), with para-toluene sulfonic acid (tosylate), with Naphthalene-2-sulfonic acid (napsylate) and with oxalic acid (oxalate).

Clopidogrel is a known pharmaceutically active compound. Clopidogrel is the dextrorotary S-enantiomer of alfa-(2-chlorophenyl)-6,7-dihydro-thieno[3,2-c]pyridine-5(4H)acetic acid-methyl ester.

The present invention also refers to a method of making these compounds and to pharmaceutically active compositions, which contain at least one compound of the present invention in a concentration known per se. The present invention further refers to the use of the new compounds and forms for the preparation of pharmaceutically active compositions, which contain at least one compound of the present invention in a pharmaceutically effective concentration.

EP 0 099 802 discloses the racemic mixture as well as both enantiomeric forms of Clopidogrel. EP 1 087 976 discloses further salts of Clopidogrel.

The present invention refers to six new polymorphic forms of (+)-(S)-Clopidogrel-hydrogenbromide, which are named herein as polymorphic “Form A”, polymorphic “Form B”, polymorphic “Form C”, polymorphic “Form D”, polymorphic “Form E”, and as polymorphic “Form F”, as well as two new polymorphic forms of (+)-(S)-Clopidogrel-napsylate, which are named herein as polymorphic “Form A” and polymorphic “Form B”. These polymorphic forms differ from each other in their powder-roentgen-diagrams (XRPD). The polymorphic forms of Clopidogrel hydrobromide in addition differ from each other by their infrared spectra. In the present description the XRPD-peaks are used for the characterization of the compounds.

The characteristic XRPD-peaks of Clopidogrel-hydrobromide of the polymorphic forms A, B, C, D, E and F and Clopidogrel napsylate of the polymorphic forms A and B are given in degree 29 with an exactness of ±0.2 degree 2Θ, and are, as listed in the following Table 1 und Table 2, at the following divergence angles.

TABLE 1

| Clopidogrel | | |
|---|---|---|
| hydrobromide Form | Angle [2Θ°]: | Relative intensity |
| A | 9.83 | medium |
| | 10.35 | medium |
| | 19.98 | strong |
| | 23.03 | strong |
| B | 9.49 | medium |
| | 10.39 | medium |
| | 12.87 | medium |
| | 19.53 | strong |
| C | 8.20 | strong |
| | 8.92 | strong |
| D | 9.76 | medium |
| | 10.40 | week-medium |
| | 19.50 | strong |
| | 23.01 | strong |
| E | 7.72 | medium |
| | 9.27 | medium |
| | 9.88 | medium |
| | 11.91 | medium |
| F | 12.48 | strong |
| | 15.89 | medium |
| | 20.16 | strong |
| | 21.97 | strong |

TABLE 2

| Clopidogrel | | |
|---|---|---|
| napsylate Form | Angle [2Θ°]: | Relative intensity |
| A | 8.59 | medium-strong |
| | 13.55 | medium-strong |
| | 19.00 | medium-strong |
| | 21.34 | strong |
| B | 7.67 | medium |
| | 8.41 | strong |
| | 9.05 | medium |
| | 10.00 | medium |

Clopidogrel hydrobromide of Form A is obtained either by combining hydrogen bromide (HBr) and Clopidogrel base in a suitable solvent and subsequent crystallization, or by recrystallization or crystal transformation from the suspension of any form of Clopidogrel hydrobromide from a suitable solvent or mixture of solvents. Suitable solvents are acetone, acetic acid ethyl ester, diisopropylether, tert.-butyl-methylether, methyl-isobutylketone, dichloromethane, toluene, isobutyronitrile, isopropanol, preferably at temperatures between 18° C. and 22° C., using a mixture of solvents containing methyl-isobutylketone und isopropanol, preferably in a weight-ratio of 4:1.

In this sense the invention refers to a method of making Clopidogrel hydrobromide of Form A, which is characterized in that Clopidogrel hydrobromide of any crystalline form is crystallized from a solvent or a mixture of solvents, comprising acetone, acetic acid ethyl ester, diisopropylether, tert.-butyl-methylether, methyl-isobutylketone, dichloromethane, toluene, isobutyronitrile, and/or isopropanol, preferably methyl-isobutylketone and/or isopropanol, preferably in a weight ratio of 4:1, within a temperature range of 18° C. to 22° C.

Clopidogrel hydrobromide of Form B is obtained by combining hydrogenbromide (HBr) and Clopidogrel base in a suitable solvent and subsequent crystallization, preferably by crystallizing from this solution by quickly crossing the saturation curves using techniques such as quick addition of an antisolvens or by evaporation crystallization, or by very quick cooling of the crystallization solution (shock cooling). Suitable solvents are acetone and dichloromethane. Suitable antisolvens are aliphatic hydrocarbons such as heptane or hexane.

The invention refers to a method of making Clopidogrel hydrobromide of Form B, which is characterized in that Clopidogrel hydrobromide of any crystalline form is crystallized from a suitable solvent, preferably acetone and/or dichloromethane, by quickly crossing the saturation curve, preferably by quick addition of an antisolvens, preferably of an aliphatic hydrocarbon, preferably heptane and/or hexane, or by evaporation crystallization, or by very quick cooling of the crystallization solution (shock cooling).

Clopidogrel hydrobromide of Form C is obtained either by combining HBr and Clopidogrel base in a suitable solvent and subsequent crystallization or by recrystallization or by crystal transformation from a suspension of any Form of Clopidogrel hydrobromide from a suitable solvent or mixture of solvents. A suitable solvent is acetonitrile.

The invention refers to a method of making Clopidogrel hydrobromide of Form C, which is characterized in that Clopidogrel hydrobromide of any crystalline Form is crystallized from acetonitrile.

Clopidogrel hydrobromide of Form D is obtained by either combining HBr and Clopidogrel base in a suitable solvent and subsequent crystallization or by recrystallization or by crystal transformation from a suspension of any Form of Clopidogrel hydrobromide from a suitable solvent or mixture of solvents, comprising acetone, acetic acid ethyl ester, diisopropylether, tert.-butyl-methylether, methyl-isobutylketone,—dichloromethane, Toluene, isobutyronitrile and/or isopropanol, preferably methyl-isobutylketone and/or isopropanol, preferably in a weight ratio of 4:1, within a temperature range from 30° C. to 60° C.

The invention refers to a method of making Clopidogrel hydrobromide of Form D, which is characterized in that Clopidogrel hydrobromide of any crystalline Form is crystallized from a solvent or a mixture of solvents, comprising acetone, acetic acid ethyl ester, diisopropylether, tert.-butyl-methylether, methyl-isobutylketone, dichloromethane, toluene, isobutyronitrile and/or isopropanol, preferably methyl-isobutylketone and/or isopropanol, preferably in a weight ratio of 4:1, within a temperature range from 30° C. to 60° C.

Clopidogrel hydrobromide of Form E is obtained either by combining of HBr and Clopidogrel base in a suitable solvent and subsequent crystallization or by crystallization of any Form of Clopidogrel hydrobromide from a suitable solvent or a mixture of solvents. Suitable solvents are mixtures of dichloromethane and aliphatic hydrocarbons. Especially preferred are long crystallization times of up to 24 hours, a working temperature range of 0° C. to 25° C. and crystallization of Form E by slow evaporation of the lower boiling solvent from the solvent mixture.

The invention refers to a method of making Clopidogrel hydrobromide of Form E, which is characterized in that Clopidogrel hydrobromide of any crystalline Form is crystallized from dichloromethane and/or an aliphatic hydrocarbon with a boiling point of preferably 60° C. to 125° C., preferably hexane, heptane or octane, preferably within a temperature range from 0° C. to 25° C., or by crystallization by slow evaporation of the lower boiling solvent from the solvent mixture at temperatures within the temperature range of 0° C. to 25° C. Preferred are long crystallization times of up to 24 hours.

Clopidogrel hydrobromide of Form F is obtained by combining HBr und Clopidogrel base in a suitable solvent and subsequent crystallization or by recrystallization of any Form of Clopidogrel hydrobromide from a suitable solvent or mixture of solvents, comprising acetone, acetic acid ethyl ester, diisopropylether, tert.-butyl-methylether, methyl-isobutylketone, dichloromethane, Toluene, isobutyronitrile and/or isopropanol. Preferred is methyl-isobutylketone and/or isopropanol, preferably in a weight ration of 4:1, whereby crystallization is carried out within a temperature range of −5° C. to +15° C. Preferred are long crystallization and stirring times of the solution and suspensions, preferably longer than 24 hours.

The invention refers to a method of making of Clopidogrel hydrobromide of Form F, which is characterized in that Clopidogrel hydrobromide of any crystalline Form is crystallized from a solvent or a mixture of solvents, comprising acetone, acetic acid ethyl ester, diisopropylether, tert.-butyl-methylether, methyl-isobutylketone, dichloromethane, toluene, isobutyronitrile and/or isopropanol, preferably methyl-isobutylketone and/or isopropanol, preferably in a weight ration of 4:1, within a temperature range of −5° C. to +15° C.

Clopidogrel also forms salts with selected organic sulfonic acids. The present invention therefore also refers to the salts Clopidogrel besylate, Clopidogrel tosylate, and Clopidogrel napsylate as Form A and Form B, as well as to Clopidogrel oxalate.

Clopidogrel besylate is obtained by combining equimolar amounts of benzenesulfonic acid and Clopidogrel base in a suitable solvent to react together. Suitable solvents are for example alcohols, ethers and/or nitrites. Preferred as a solvent is methanol. Preferably the compound is isolated by solvent abstraction, i.e. for example by removing the solvent by distillation or by spray drying.

Clopidogrel tosylate is obtained by combining equimolar amounts of para-toluenesulfonic acid with Clopidogrel base in a suitable solvent to react together. Suitable solvents are for example alcohols, ethers and/or nitrites. Preferred as a solvent is methanol at a working temperature of 20-25° C. Preferably the compound is isolated by solvent abstraction.

Clopidogrel napsylate Form A is obtained by combining equimolar amounts of naphthalene-2-sulfonic acid with Clopidogrel base in a suitable solvent and initiating crystallization by inoculating the crystallization solution with Clopidogrel napsylate Form A. Suitable solvents are for example primary and secondary alcohols, ethers, nitrites, toluene and aqueous solvent mixtures, preferably thereof, with a water content of preferably less than 10% by weight (<10% by weight). The suitable temperature working range is between 20° C. and 60° C. Preferred solvents are isopropanol, water, diisopropylether, especially preferred is isopropanol. Alternatively Clopidogrel napsylate Form A is obtained from other Clopidogrel salts (e.g. from Clopidogrel hydrobromide) by salt transformation in the presence of naphthalene-2-sulfonic acid salts (e.g. sodium-2-naphthylsulfonate). Suitable solvents are: isopropanol, diisopropylether, and aqueous solvent mixtures, preferably thereof, with a water content of preferably less than 10% by weight of water. The preferred working temperature range is also here 20° C. to 60° C.

Clopidogrel napsylate Form A is obtained directly and without inoculation, by reacting equimolar amounts of naphthalene-2-sulfonic acid with Clopidogrel base in a suitable solvent, as described in the foregoing paragraph, wherein said naphthalene-2-sulfonic acid has a purity of at least 99.5% by weight and preferably, wherein the content of sulfonic-1-sulfonic acid is less than 0.5% by weight.

Clopidogrel napsylate Form B is obtained by dissolving equimolar amounts of naphthalene-2-sulfonic acid with Clopidogrel base in a suitable solvent and initiating crystallization by inoculation with Clopidogrel napsylate Form B. Suitable solvents are primary and secondary alcohols, nitrites, toluene and/or aqueous solvent mixtures, preferably thereof, with a water content of preferably less than 10% by weight of water. Especially preferred is isopropanol as a solvent, a strongly over saturated crystallizing solution (>20%), a temperature working range from 15° C. to 20° C., as well as prolonged mixing times of up to 24 hours (crystallization and mixing of the suspension).

Alternatively Clopidogrel napsylate Form B is also obtained by salt transformation from other Clopidogrel salts (e.g. Clopidogrel hydrobromide) in the presence of naphthalene-2-sulfonic acid salts (e.g. sodium-2-naphthylsulfonate) as well as by recrystallization from Clopidogrel napsylate Form A by inoculating the solution with the Form B. Suitable solvents are isopropanol, diisopropylether, and aqueous solvent mixtures, preferably thereof, with a water content of preferably less than 10% by weight (<10% by weight) water, at a preferred temperature working range of 15° C. to 20° C. as well as prolonged mixing times of up to 24 hours (crystallizing and mixing of the suspension).

Clopidogrel napsylate Form B is obtained directly without inoculation by reacting equimolar amounts of naphthalene-2-sulfonic acid with Clopidogrel base in a suitable solvent, as described herein before, wherein the naphthalene-2-sulfonic acid used has a purity of less than 99.0% by weight and especially, if its content of naphthalene-1-sulfonic acid is higher than 1.0% by weight.

The present invention refers to the compound Clopidogrel oxalate. Clopidogrel oxalate is obtained by reacting equimolar amounts of oxalic acid with Clopidogrel base in a suitable solvent. Suitable solvents are for example alcohols, ethers, nitrites, and/or aqueous solvent mixtures with a water content of preferably less than 10% by weight of water. Preferred solvents are isopropanol, diisopropylether and solvent mixtures, preferably thereof, with a water content of preferably less than 10% by weight of water (<10% by weight). Preferably the compound is isolated by solvent abstraction. In the previous cases mentioned, the condition that the water content is less than 10% by weight is a preferred but not a critical limitation.

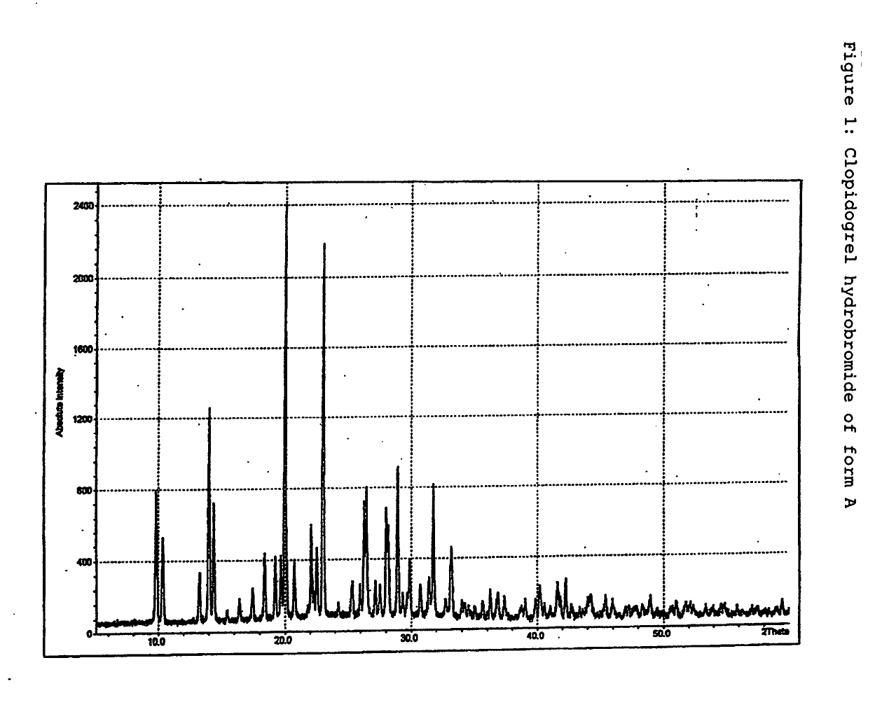

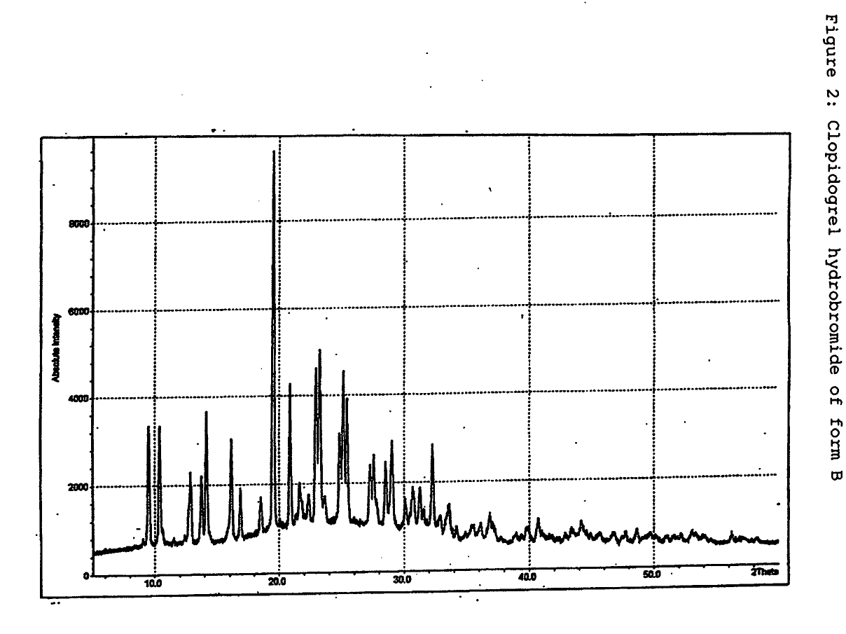

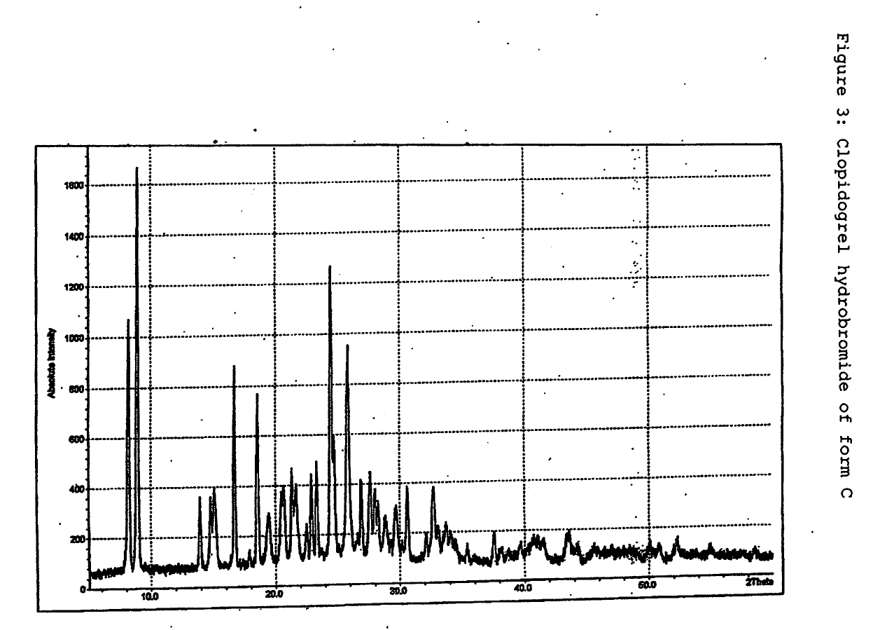

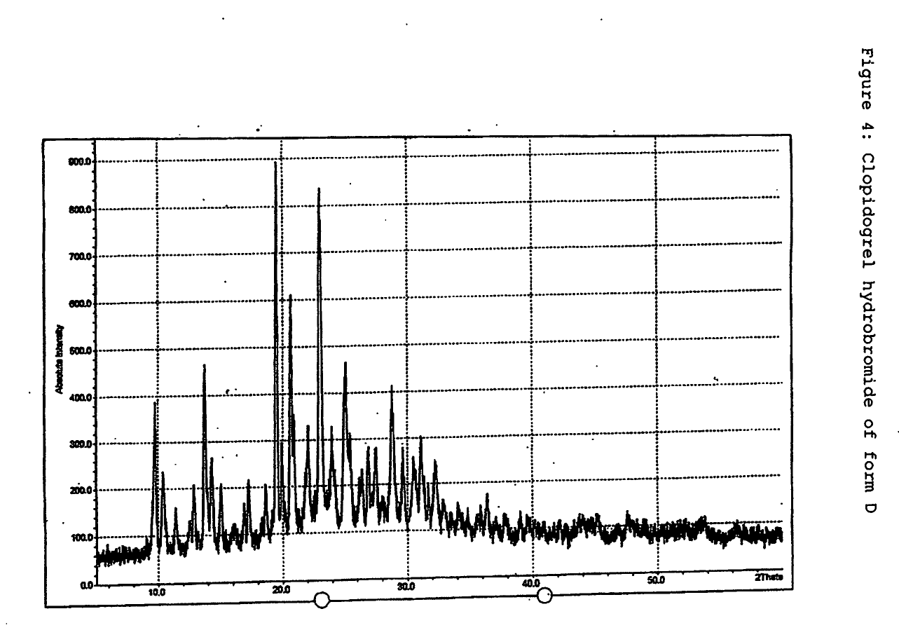

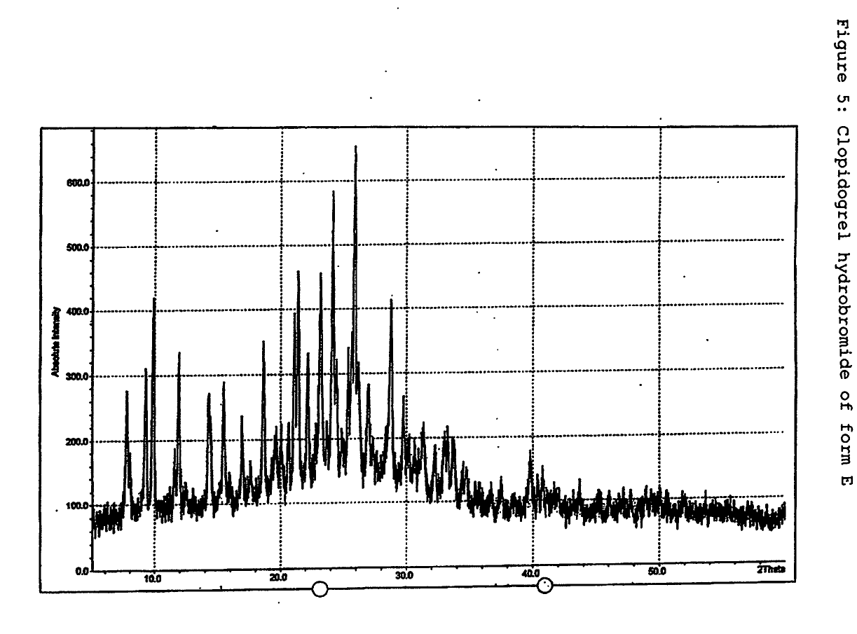

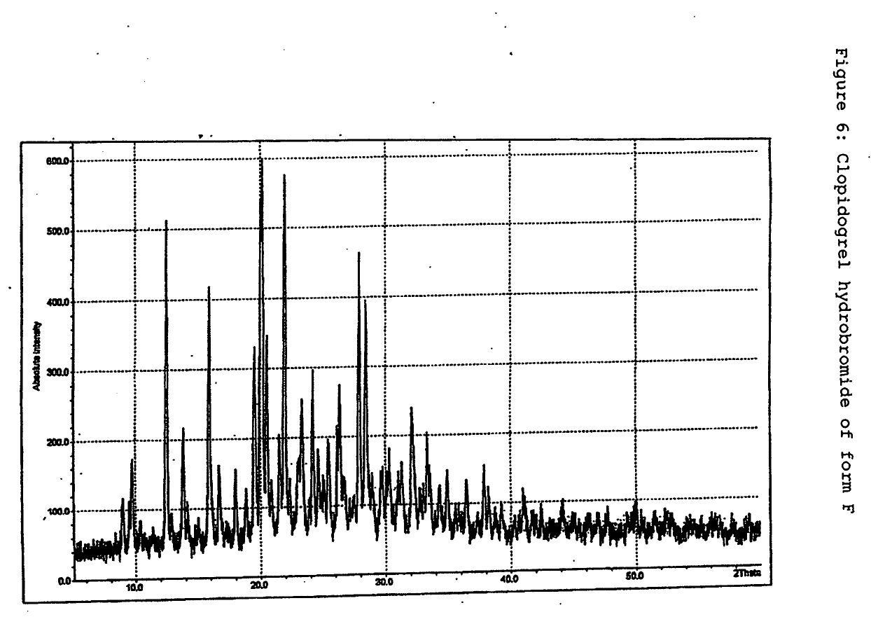

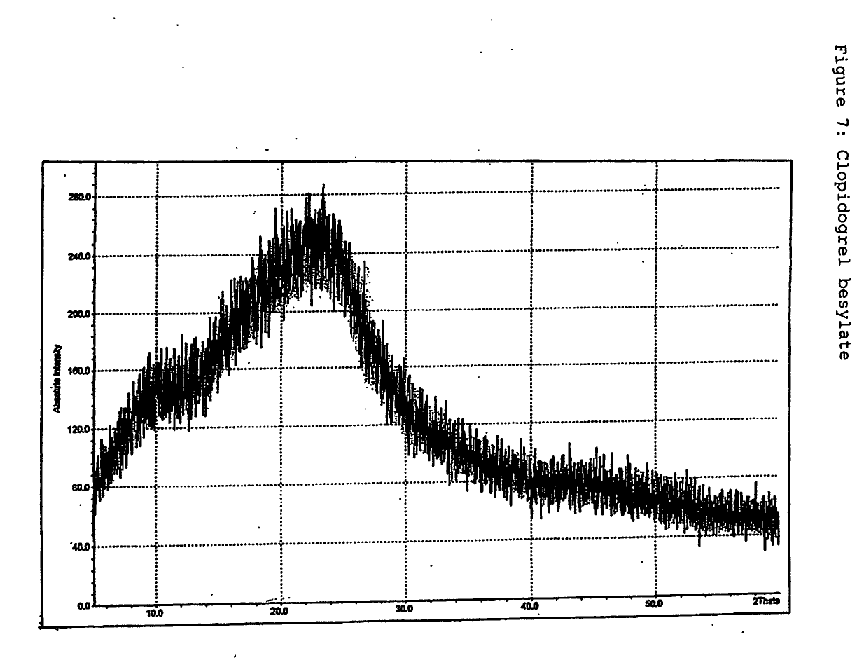

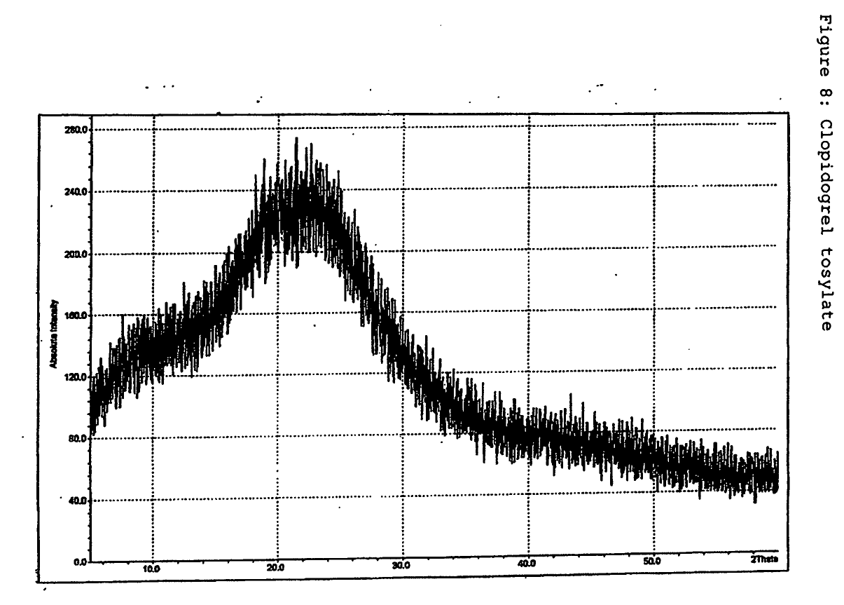

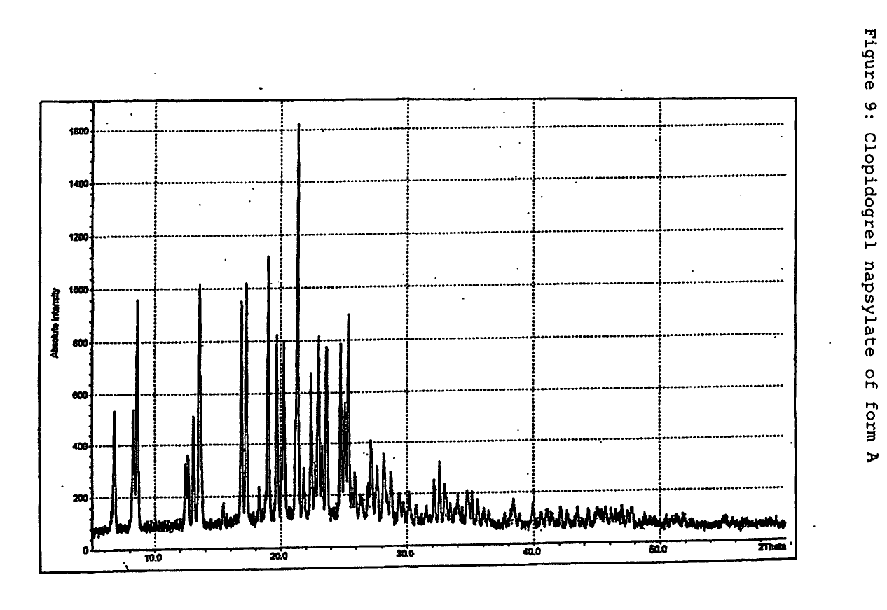

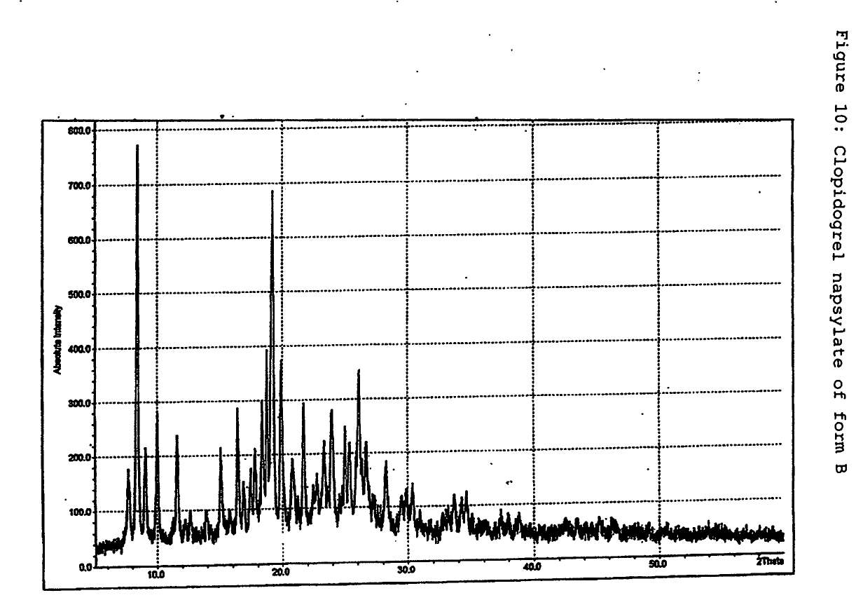

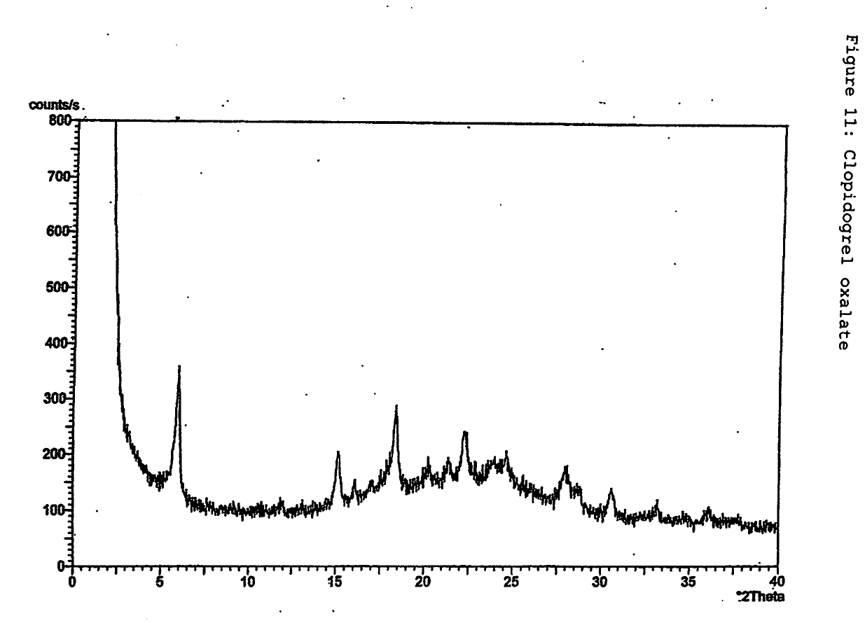

FIGS. 1-11 show the XRPD diagram of Clopidogrel HBr Form A (FIG. 1), Form B (FIG. 2), Form C (FIG. 3), Form D (FIG. 4), Form E (FIG. 5), Form F (FIG. 6), Clopidogrel besylate (FIG. 7), Clopidogrel tosylate (FIG. 8), Clopidogrel napsylate Form A (FIG. 9), Clopidogrel napsylate Form B (FIG. 10) und Clopidogrel oxalate (FIG. 11). The following examples illustrate the invention.

### EXAMPLE 1

### Clopidogrel Hydrobromide of Form A

160 g Clopidogrel base are dissolved in 260 g acetone. Hydrogen bromide gas (HBr) is being introduced into this solution under ice cooling (inside temperature: 0° C.-5° C.) until the pH-value of the solution (measured with humid indicator paper) is 2 (two). The formed suspension is left to warm up to 20° C. and is stirred for two hours. The solid is isolated using vacuum filtration and is washed with cold acetone. The humid product is dried under vacuum until it shows a constant weight. There are obtained 130 g Clopidogrel hydrobromide of Form A with the following properties:

HPLC content of Clopidogrel HBr: 100%

DSC: endothermic-maximum: 143° C.

IR (KBr pressed mass) [cm⁻¹ at % transmission]:

| | | | | | | | | | |
|---|---|---|---|---|---|---|---|---|---|
| 3484 | 67%; | 3075 | 76%; | 3005 | 58%; | 2952 | 50%; | 2704 | 59%; |
| 2628 | 46%; | 2476 | 21%; | 1753 | 3%; | 1593 | 73%; | 1474 | 37%; |
| 1437 | 17%; | 1404 | 37%; | 1349 | 42%; | 1319 | 18%; | 1297 | 20%; |
| 1226 | 8%; | 1180 | 22%; | 1135 | 55%; | 1056 | 37%; | 983 | 59%; |
| 965 | 45%; | 919 | 65%; | 885 | 75%; | 845 | 46%; | 789 | 61%; |
| 762 | 24%; | 740 | 30%; | 706 | 51%; | 626 | 86%; | 597 | 72%; |
| 534 | 78%; | 454 | 70%. | | | | | | |

XRPD [Cu Kα₁]:

| Angle [2Θ°]: | Relative intensity [%] |
|---|---|
| 9.83 | 33 |
| 10.35 | 22 |
| 13.24 | 14 |
| 14.01 | 51 |
| 14.37 | 30 |
| 16.40 | 8 |
| 17.44 | 10 |
| 18.39 | 18 |
| 19.22 | 18 |
| 19.68 | 18 |
| 19.98 | 100 |
| 20.73 | 16 |
| 22.08 | 25 |
| 22.53 | 19 |
| 23.03 | 90 |
| 25.93 | 11 |
| 26.26 | 30 |
| 26.44 | 34 |
| 27.13 | 11 |
| 27.49 | 11 |
| 28.01 | 28 |
| 28.91 | 37 |
| 29.29 | 8 |
| 29.85 | 16 |
| 30.71 | 10 |
| 31.42 | 12 |
| 31.75 | 34 |
| 33.17 | 19 |
| 36.22 | 9 |
| 37.33 | 7 |
| 40.16 | 9 |
| 41.58 | 10 |
| 42.23 | 10 |
| 48.92 | 7 |

### EXAMPLE 2

### Clopidogrel Hydrobromide of Form B

10 g Clopidogrel hydrobromide are dissolved in 60 g acetone whereby the mixture is mildly warmed up until complete solution of the compound. The solution is evacuated under stirring in a large round bottom flask. A white residue of 10 g of Clopidogrel hydrobromide of the amorphous Form B is obtained with the following properties:

HPLC content of Clopidogrel HBr: 100%

DSC: endothermic-maximum: week minimum at about 130° C.

IR (KBr pressed mass) [cm⁻¹ at % transmission]:

| | | | | | | | | | |
|---|---|---|---|---|---|---|---|---|---|
| 3436 | 39%; | 2952 | 50%; | 2479 | 27%; | 1754 | 3%; | 1708 | 50%; |
| 1636 | 69%; | 1480 | 38%; | 1437 | 13%; | 1320 | 26%; | 1296 | 26%; |
| 1224 | 13%; | 1179 | 25%; | 1134 | 64%; | 1056 | 46 | 1038 | 44%; |
| 1011 | 47%; | 963 | 63%; | 917 | 78%; | 883 | 76%; | 843 | 60%; |
| 788 | 68%; | 762 | 26%; | 727 | 41%; | 627 | 79%; | 597 | 65%; |
| 531 | 76%; | 455 | 67%. | | | | | | |

XRPD [Cu Kα₁]:

| Angle [2Θ°]: | Relative intensity [%] |
|---|---|
| 9.50 | 34.95 |
| 10.39 | 34.57 |
| 12.87 | 24.42 |
| 13.74 | 23.08 |
| 14.14 | 38.5 |
| 16.13 | 31.84 |
| 16.86 | 20.24 |
| 18.52 | 18.04 |
| 19.53 | 100 |
| 20.88 | 44.26 |
| 21.63 | 20.92 |
| 22.34 | 18.09 |
| 22.93 | 47.93 |
| 23.23 | 52.29 |
| 23.60 | 17.76 |
| 24.83 | 32.92 |
| 25.12 | 47.4 |
| 25.41 | 40.78 |
| 27.25 | 24.32 |
| 27.54 | 26.55 |
| 28.50 | 25.57 |
| 29.01 | 30.56 |
| 30.07 | 16.68 |
| 30.67 | 19.36 |
| 31.23 | 19.37 |
| 31.53 | 14.47 |
| 32.26 | 29.23 |
| 33.57 | 15.51 |
| 34.16 | 10.02 |
| 36.09 | 10.93 |
| 36.83 | 12.91 |
| 40.70 | 11.28 |
| 44.15 | 11.06 |
| 48.63 | 8.98 |
| 9.50 | 34.95 |

### EXAMPLE 3

### Clopidogrel Hydrobromide of Form C

13 g Clopidogrel hydrobromide are stirred in 30 ml acetonitrile for several hours at room temperature. The solid material is then isolated by vacuum filtration. The humid material is dried under vacuum until a constant weight. 11 g Clopidogrel hydrobromide of Form C are obtained having the following properties:

HPLC content of Clopidogrel HBr: 100%

DSC: endothermic-maximum: 145° C.

IR (KBr pressed mass) [cm⁻¹ at % transmission]:

| | | | | | | | | | |
|---|---|---|---|---|---|---|---|---|---|
| 3437 | 65%; | 3064 | 48%; | 3003 | 56%; | 2952 | 51%; | 2910 | 51%; |
| 2533 | 24%; | 1758 | 3%; | 1593 | 77%; | 1480 | 44%; | 1439 | 21%; |
| 1392 | 47%; | 1348 | 44%; | 1320 | 32%; | 1295 | 12%; | 1217 | 17%; |
| 1178 | 18%; | 1071 | 51%; | 1031 | 44%; | 1015 | 43%; | 973 | 59%; |
| 952 | 63%; | 911 | 72%; | 891 | 69%; | 838 | 65%; | 784 | 76%; |
| 756 | 22%; | 712 | 33%; | 624 | 68%; | 591 | 71%; | 536 | 84%; |
| 456 | 74%. | | | | | | | | |

XRPD [Cu Kα₁]:

| Angle [2Θ°]: | Relative intensity [%] |
|---|---|
| 8.20 | 63 |
| 8.92 | 100 |
| 13.91 | 21 |
| 14.76 | 21 |
| 15.07 | 22 |
| 16.67 | 52 |
| 18.52 | 45 |
| 19.42 | 17 |
| 20.49 | 22 |
| 21.31 | 27 |
| 21.62 | 23 |
| 22.49 | 14 |
| 22.88 | 25 |
| 23.31 | 28 |
| 24.46 | 74 |
| 25.83 | 55 |
| 26.87 | 25 |
| 27.60 | 25 |
| 27.96 | 21 |
| 28.81 | 15 |
| 29.66 | 18 |
| 30.60 | 22 |
| 32.67 | 22 |
| 37.51 | 11 |

### EXAMPLE 4

### Clopidogrel Hydrobromide of Form D

1 g Clopidogrel hydrobromide is stirred over night in 2 ml isopropanol at 40° C. The solid material is then isolated using vacuum filtration. The humid material is then dried under vacuum until constant weight. There are obtained 0.8 g Clopidogrel hydrobromide of Form D with the following properties:

HPLC content of Clopidogrel HBr: 100%

DSC: endothermic-maximum: 144° C.

IR (KBr pressed mass) [cm⁻¹ at % transmission]:

| | | | | | | | | | |
|---|---|---|---|---|---|---|---|---|---|
| 3483 | 58%; | 3110 | 78%; | 3075 | 82%; | 3021 | 79%; | 2906 | 61%; |
| 2486 | 30%; | 2362 | 34%; | 1753 | 3%; | 1484 | 58%; | 1436 | 29%; |
| 1391 | 47%; | 1337 | 51%; | 1316 | 46%; | 1295 | 22%; | 1260 | 47%; |
| 1228 | 19%; | 1188 | 35%; | 1136 | 72%; | 1061 | 57%; | 1035 | 51%; |
| 1009 | 45%; | 967 | 66%; | 944 | 63%; | 903 | 72%; | 845 | 69%; |
| 787 | 84%; | 748 | 39%; | 733 | 38%; | 708 | 52%; | 622 | 82%; |
| 597 | 76%; | 542 | 91%; | 484 | 87%; | 454 | 80%. | | |

XRPD [Cu Kα₁]:

| Angle [2Θ°]: | Relative intensity [%] |
|---|---|
| 9.76 | 43 |
| 10.40 | 10 |
| 11.38 | 11 |
| 12.85 | 13 |
| 13.73 | 52 |
| 14.30 | 27 |
| 15.02 | 22 |
| 17.23 | 24 |
| 19.50 | 100 |
| 19.91 | 33 |
| 20.65 | 68 |
| 22.03 | 29 |
| 23.01 | 95 |
| 23.97 | 35 |
| 25.07 | 52 |
| 26.86 | 31 |
| 27.45 | 30 |
| 28.76 | 44 |
| 29.63 | 30 |
| 31.10 | 32 |

### EXAMPLE 5

### Clopidogrel Hydrobromide of Form E

13.5 g Clopidogrel hydrobromide are dissolved in 140 g dichloromethane. 82 g heptane (isomeric mixture) are added to the solution at room temperature and stirred over night under nitrogen gas. The solid material is isolated from the suspension obtained using vacuum filtration and is dried until constant weight. 13 g Clopidogrel hydrobromide of Form E are obtained having the following properties:

HPLC content of Clopidogrel HBr: 100%

DSC: endothermic-maximum: 125° C.

IR (KBr pressed mass) [cm⁻¹ at % transmission]:

| | | | | | | | | | |
|---|---|---|---|---|---|---|---|---|---|
| 3485 | 57%; | 3007 | 64%; | 2956 | 44%; | 2908 | 41%; | 2489 | 19%; |
| 1748 | 3%; | 1593 | 75%; | 1481 | 40%; | 1438 | 18%; | 1397 | 46%; |
| 1345 | 42%; | 1321 | 31%; | 1297 | 13%; | 1263 | 43%; | 1229 | 12%; |
| 1180 | 26%; | 1059 | 52%; | 1034 | 43%; | 1015 | 33%; | 968 | 65%; |
| 951 | 64%; | 909 | 72%; | 892 | 71%; | 841 | 60%; | 786 | 72%; |
| 758 | 24%; | 720 | 17%; | 623 | 72%; | 593 | 73%; | 539 | 87%; |
| 480 | 81%; | 456 | 73%; | 421 | 86%. | | | | |

XRPD [Cu Kα₁]:

| Angle [2Θ°]: | Relative intensity [%] |
|---|---|
| 7.72 | 41 |
| 9.27 | 47 |
| 9.88 | 65 |
| 11.91 | 51 |
| 14.28 | 41 |
| 15.45 | 42 |
| 16.91 | 34 |
| 20.65 | 32 |
| 21.10 | 59 |
| 21.38 | 71 |
| 22.17 | 50 |
| 23.15 | 68 |
| 24.11 | 86 |
| 25.36 | 52 |
| 25.87 | 100 |
| 26.96 | 43 |
| 28.74 | 64 |
| 29.74 | 39 |

### EXAMPLE 6

### Clopidogrel Hydrobromide of Form F

A mixture of 3500 g isopropanol and 620 g Clopidogrel hydrobromide of Form A are heated until a clear, slightly yellow solution is obtained (inside temperature (IT): 60° C.-65° C.). After quick cooling to an inside temperature of 10° C. there crystallizes spontaneously, optionally after inoculation, a white powdery mass, which is isolated by vacuum filtration and is dried until constant weight. 361 g Clopidogrel hydrobromide of Form F are obtained having the following properties: HPLC content of Clopidogrel HBr: 100%; DSC: endothermic-maximum: 107.6° C.

IR (KBr pressed mass) [cm⁻¹ at % transmission]:

| | | | | | | | | | |
|---|---|---|---|---|---|---|---|---|---|
| 3325 | 16% | 3113 | 46% | 3067 | 61% | 3013 | 53% | 3001 | 51% |
| 2961 | 50% | 2889 | 57% | 2858 | 57% | 2725 | 55% | 2479 | 37% |
| 2349 | 57% | 2299 | 60% | 2142 | 66% | 1956 | 81% | 1744 | 3% |
| 1613 | 58% | 1588 | 63% | 1573 | 77% | 1493 | 49% | 1470 | 26% |
| 1453 | 26% | 1434 | 19% | 1423 | 15% | 1390 | 52% | 1364 | 60% |
| 1351 | 41% | 1334 | 30% | 1322 | 28% | 1285 | 29% | 1276 | 33% |
| 1257 | 29% | 1239 | 23% | 1222 | 29% | 1211 | 19% | 1188 | 30% |
| 1171 | 23% | 1093 | 66% | 1056 | 30% | 1043 | 39% | 1028 | 41% |
| 1011 | 28% | 984 | 62% | 965 | 57% | 955 | 60% | 930 | 73% |
| 918 | 78% | 906 | 57% | 877 | 75% | 865 | 69% | 842 | 48% |
| 826 | 77% | 786 | 53% | 762 | 8% | 729 | 19% | 715 | 44% |
| 672 | 82% | 637 | 70% | 598 | 47% | 590 | 43% | 530 | 42% |
| 505 | 58% | 485 | 59% | 457 | 47% | 434 | 76% | 425 | 69% |

XRPD [Cu Kα₁]:

| Angle [2Θ°]: | Relative intensity [%] |
|---|---|
| 8.95 | 19 |
| 9.74 | 27 |
| 12.48 | 82 |
| 13.83 | 34 |
| 15.89 | 66 |
| 16.67 | 28 |
| 17.99 | 25 |
| 18.84 | 20 |
| 19.53 | 54 |
| 20.02 | 80 |
| 20.16 | 100 |
| 20.52 | 56 |
| 20.86 | 21 |
| 21.52 | 33 |
| 21.97 | 94 |
| 22.32 | 22 |
| 23.35 | 42 |
| 24.20 | 45 |
| 24.65 | 18 |
| 25.46 | 32 |
| 26.16 | 36 |
| 26.36 | 45 |
| 27.91 | 73 |
| 28.44 | 54 |
| 31.28 | 25 |
| 32.14 | 28 |
| 33.33 | 31 |
| 34.91 | 25 |
| 36.43 | 12 |
| 37.85 | 16 |
| 41.01 | 13 |

### EXAMPLE 7

### Clopidogrel Besylate

3.0 g benzenesulfonic acid and 5.5 g Clopidogrel base are dissolved in 30 ml methanol. The solvent is evaporated in vacuum. 8.5 g Clopidogrel besylate are obtained with the following properties:

HPLC content of Clopidogrel besylate: 100%

DSC: endothermic-maximum: none

IR (KBr pressed mass) [cm⁻¹ at % transmission]:

| | | | | | | | | | |
|---|---|---|---|---|---|---|---|---|---|
| 3437 | 28%; | 3066 | 56%; | 2957 | 42%; | 2579 | 44%; | 1752 | 3%; |
| 1636 | 65%; | 1593 | 76%; | 1479 | 31%; | 1444 | 14%; | 1322 | 36%; |
| 1226 | 3%; | 1159 | 3%; | 1122 | 4%; | 1069 | 32%; | 1034 | 11%; |
| 1016 | 6%; | 996 | 14%; | 913 | 69%; | 887 | 70%; | 840 | 67%; |
| 759 | 16%; | 727 | 10%; | 694 | 20%; | 611 | 4%; | 565 | 26%; |
| 480 | 76%; | 457 | 74%. | | | | | | |

XRPD [Cu Kα₁]: no clear peaks detectable

### EXAMPLE 8

### Clopidogrel Tosylate

3.2 g para-toluenesulfonic acid and 5.5 g Clopidogrel base are dissolved in 30 ml methanol. The solvent is evaporated by vacuum. There remain 8.7 g Clopidogrel tosylate with the following properties:

HPLC content of Clopidogrel besylate: 100%

DSC: endothermic-maximum: none

IR (KBr pressed mass) [cm⁻¹ at % transmission]:

XRPD [Cu Kα₁]: no clear peaks detectable

### EXAMPLE 9

### Clopidogrel Napsylate, Form A

52.5 g sodium-2-naphthylsulfonate are dissolved in 430 ml dematerialized water under heating at about 75° C. A solution of 50 g Clopidogrel hydrogen sulfate in 200 ml water is added to the solution. The resulting mixture is cooled to room temperature and the upper oily phase is separated. The separated oil is dissolved in 230 g isopropanol. The obtained solution is dried with magnesium sulfate and diluted with 250 g diisopropylether. The solution is inoculated at a temperature of about 60° C. with Clopidogrel napsylate and stirred over night whilst cooling to room temperature. The solid material is isolated by vacuum filtration, washed with diisopropylether and dried under vacuum. 37 g Clopidogrel napsylate of Form A are obtained with the following properties:

HPLC content of Clopidogrel napsylate: 100%

DSC: endothermic maximum: 149° C.

IR (KBr pressed mass) [cm⁻¹ at % transmission]:

| | | | | | | | | | |
|---|---|---|---|---|---|---|---|---|---|
| 3438 | 57%; | 2969 | 47%; | 2672 | 63%; | 2593 | 59%; | 2362 | 72%; |
| 1751 | 10%; | 1595 | 79%; | 1475 | 54%; | 1438 | 53%; | 1329 | 54%; |
| 1301 | 59%; | 1222 | 11%; | 1171 | 3%; | 1135 | 29%; | 1090 | 21%; |
| 1032 | 10%; | 993 | 60%; | 956 | 78%; | 906 | 82%; | 886 | 83%; |
| 866 | 74%; | 830 | 64%; | 783 | 83%; | 753 | 27%; | 724 | 76%; |
| 698 | 48%; | 676 | 21%; | 650 | 71%; | 623 | 73%; | 597 | 76%; |
| 567 | 47%; | 480 | 69%; | 461 | 76%; | 421 | 78%. | | |

XRPD [Cu Kα₁]:

| Angle [2Θ°]: | Relative intensity [%] |
|---|---|
| 6.79 | 32 |
| 8.27 | 33 |
| 8.59 | 59 |
| 12.44 | 21 |
| 12.62 | 22 |
| 13.07 | 31 |
| 13.55 | 62 |
| 16.87 | 59 |
| 17.24 | 63 |
| 18.25 | 14 |
| 19.00 | 71 |
| 19.69 | 52 |
| 20.02 | 19 |
| 20.24 | 47 |
| 21.34 | 100 |
| 21.82 | 17 |
| 22.40 | 42 |
| 22.72 | 19 |
| 23.02 | 50 |
| 23.27 | 25 |
| 23.65 | 47 |
| 24.75 | 49 |
| 25.09 | 33 |
| 25.34 | 56 |
| 25.85 | 18 |
| 27.11 | 25 |
| 27.61 | 19 |
| 28.12 | 22 |
| 32.14 | 15 |
| 32.55 | 20 |
| 32.97 | 14 |
| 35.10 | 11 |

### EXAMPLE 10

### Clopidogrel Napsylate, Form A

2.5 g sodium-2-naphthylsulfonate are dissolved in 60 ml of water. Suspended material is separated by filtration. 30 ml methanol and 2.9 g Clopidogrel hydrobromide are then added. The solution obtained is vigorously stirred and put under slight vacuum and kept at room temperature until about 50% by weight of the solvent has slowly evaporated. The white solid thus formed is isolated by vacuum filtration, washed with water and dried under vacuum until constant weight. 3 g Clopidogrel napsylate of Form A are obtained.

### EXAMPLE 11

### Clopidogrel Napsylate, Form B

A previously prepared hot solution (about 65° C.) of 82 g Clopidogrel napsylate Form A in 462 g isopropanol is cooled to 20-25° C. and inoculated with Clopidogrel napsylate Form B. The mixture is well stirred during 24 hours at 15-20° C. The solid is then isolated from the suspension by vacuum filtration. The filter cake is washed with isopropanol at 15-20° C. and dried in air at an inside temperature of 20-25° C. until constant weight is obtained. 70 g Clopidogrel napsylate, Form B, are obtained.

DSC: endothermic-maximum: 114.4° C.

XRPD [Cu Kα₁]:

| Angle [2Θ°] | Relative intensity [%] |
|---|---|
| 7.67 | 21 |
| 8.41 | 100 |
| 9.05 | 27 |
| 10.00 | 34 |
| 11.58 | 30 |
| 15.03 | 25 |
| 16.39 | 35 |
| 16.86 | 18 |
| 17.41 | 20 |
| 17.75 | 26 |
| 18.35 | 36 |
| 18.75 | 48 |
| 19.21 | 85 |
| 19.91 | 47 |
| 20.81 | 23 |
| 21.70 | 37 |
| 22.78 | 21 |
| 23.33 | 27 |
| 23.95 | 36 |
| 25.01 | 30 |
| 25.35 | 27 |
| 25.95 | 27 |
| 26.13 | 45 |
| 26.69 | 27 |
| 28.29 | 23 |
| 30.36 | 17 |
| 33.65 | 15 |
| 34.62 | 16 |

### EXAMPLE 12

### Clopidogrel Oxalate

10 g Clopidogrel base and 3.1 g oxalic acid are dissolved in 100 ml dichloromethane. The solvent is evaporated under vacuum. There remains 13 g Clopidogrel oxalate with the following properties:

HPLC content of Clopidogrel oxalate: 100%

DSC: endothermic-maximum: none

[symbol]

Raman [cm−1, intensity]:

| | | | | | | | | |
|---|---|---|---|---|---|---|---|---|
| 1737.5 | week | 1621.8 | medium | 1594.1 | week | 1576.0 | week | 1 [symbol] |
| 1514.5 | medium | 1498.7 | medium | 1451.5 | medium | 1396.7 | week | 1 [symbol] |
| 1329.7 | week | 1316.3 | week | 1281.7 | week | 1252.5 | week | 1 [symbol] |
| 1192.9 | week | 1167.5 | week | 1135.3 | week | 1089.5 | week | 1 [symbol] |
| 1004.6 | week | 917.9 | week | 867.7 | week | 847.6 | medium | [symbol] |
| 785.9 | week | 764.0 | week | 718.4 | medium | 687.9 | week | [symbol] |
| 670.3 | week | 635.1 | medium | 609.5 | week | 584.9 | week | [symbol] |
| 542.7 | week | 534.5 | week | 506.0 | week | 486.8 | week | [symbol] |
| 432.1 | week | 410.3 | week | | | | | |

XRPD [Cu Kα₁]: no distinct peaks are obtained.

### EXAMPLE 13

### Clopidogrel Napsylate, Form A

170 g Clopidogrel base and 115 g naphthalene-2-sulfonic acid monohydrate are dissolved in 600 ml isopropanol at a 60° C. and slowly cooled. At 50° C. the clear solution is inoculated with Clopidogrel napsylate of Form A and further cooled at a rate of 10° C./h to room temperature. The crystals are isolated by vacuum filtration and dried under vacuum. There are obtained 223 g Clopidogrel napsylate of Form A.

1. Polymorphic forms of (+)-(S)-Clopidogrel-hydrogenbromide, which are named herein as polymorphic “Form A”, polymorphic “Form B”, polymorphic “Form C”, polymorphic “Form D”, polymorphic “Form E”, and as polymorphic “Form F”, and which differ from each other in their powder-roentgen-diagrams (XRPD), according to the characteristic peaks as listed in Table 1, given in degree 2Θ with an exactness of ±0.2 Grad 2Θ:
TABLE 1

| Clopidogrel | | |
|---|---|---|
| hydrobromide Form | Angle [2Θ°]: | Relative intensity |
| A | 9.83 | medium |
| | 10.35 | medium |
| | 19.98 | strong |
| | 23.03 | strong |
| B | 9.49 | medium |
| | 10.39 | medium |
| | 12.87 | medium |
| | 19.53 | strong |
| C | 8.20 | strong |
| | 8.92 | strong |
| D | 9.76 | medium |
| | 10.40 | week-medium |
| | 19.50 | strong |
| | 23.01 | strong |
| E | 7.72 | medium |
| | 9.27 | medium |
| | 9.88 | medium |
| | 11.91 | medium |
| F | 12.48 | strong |
| | 15.89 | medium |
| | 20.16 | strong |
| | 21.97 | strong |

2. Polymorphic forms of (+)-(S)-Clopidogrel-napsylate, which are named herein as polymorphic “Form A” and polymorphic “Form B” and which differ from each other in their powder-roentgen-diagrams (XRPD), according to the characteristic peaks as listed in Table 2, given in degree 2Θ with an exactness of ±2 Grad 2Θ:
TABLE 2

| Clopidogrel | | |
|---|---|---|
| napsylate Form | Angle [2Θ°]: | Relative intensity |
| A | 8.59 | medium-strong |
| | 13.55 | medium-strong |
| | 19.00 | medium-strong |
| | 21.34 | strong |
| B | 7.67 | medium |
| | 8.41 | strong |
| | 9.05 | medium |
| | 10.00 | medium |

3. Method of making Clopidogrel hydrobromide of Form A according to claim 1, characterized in that Clopidogrel hydrobromide of any crystalline form is crystallized from a solvent or a mixture of solvents, comprising acetone, acetic acid ethyl ester, diisopropylether, tert.-butyl-methylether, methyl-isobutylketone, dichloromethane, toluene, isobutyronitrile, and/or isopropanol, preferably methyl-isobutylketone and/or isopropanol, preferably in a weight ratio of 4:1, within a temperature range of 18° C. to 22° C.

4. Method of making Clopidogrel hydrobromide of Form B according to claim 1, characterized in that Clopidogrel hydrobromide of any crystalline form is crystallized from a suitable solvent, preferably acetone and/or dichloromethane, by quickly crossing the saturation curve, preferably by quick addition of an antisolvens, preferably of an aliphatic hydrocarbon, preferably heptane and/or hexane, or by evaporation crystallization, or by very quick cooling of the crystallization solution (shock cooling).

5. Method of making Clopidogrel hydrobromide of Form C according to claim 1, characterized in that Clopidogrel hydrobromide of any crystalline Form is crystallized from acetonitrile.

6. Method of making Clopidogrel hydrobromide of Form D according to claim 1, characterized in that Clopidogrel hydrobromide of any crystalline Form is crystallized from a solvent or a mixture of solvents, comprising acetone, acetic acid ethyl ester, diisopropylether, tert.-butyl-methylether, methyl-isobutylketone, dichloromethane, toluene, isobutyronitrile and/or isopropanol, preferably methyl-isobutylketone and/or isopropanol, preferably in a weight ratio of 4:1, within a temperature range from 30° C. to 60° C.

7. Method of making Clopidogrel hydrobromide of Form E according to claim 1, characterized in that Clopidogrel hydrobromide of any crystalline Form is crystallized from dichloromethane and/or an aliphatic hydrocarbon with a boiling point of preferably 60° C. to 125° C., preferably hexane, heptane or octane, preferably within a temperature range from 0° C. to 25° C., or by crystallization by slow evaporation of the lower boiling solvent from the solvent mixture at temperatures within the temperature range of 0° C. to 25° C., preferably at long crystallization times of up to 24 hours.

8. Method of making of Clopidogrel hydrobromide of Form F according to claim 1, characterized in that Clopidogrel hydrobromide of any crystalline Form is crystallized from a solvent or a mixture of solvents, comprising acetone, acetic acid ethyl ester, diisopropylether, tert.-butyl-methylether, methyl-isobutylketone, dichloromethane, toluene, isobutyronitrile and/or isopropanol, preferably methyl-isobutylketone and/or isopropanol, preferably in a weight ration of 4:1, within a temperature range of −5° C. to +15° C.

9. The salts Clopidogrel besylate, Clopidogrel tosylate and Clopidogrel oxalate.

10. Method of making Clopidogrel besylate according to claim 9, characterized in that equimolar amounts of benzenesulfonic acid and Clopidogrel base are combined in a suitable solvent to react together, preferably in an alcohol, ether and/or nitrile, preferably in methanol, whereby the compound is preferably isolated by solvent abstraction, preferably by removing the solvent by distillation or by spray drying.

11. Method for making Clopidogrel tosylate according to claim 9, characterized in that equimolar amounts of para-toluenesulfonic acid are combined with Clopidogrel base in a suitable solvent to react together, preferably in an alcohol, ether and/or nitrile, preferably in methanol, preferably at a working temperature of 20-25° C., whereby the compound is preferably isolated by solvent abstraction.

12. Method of making Clopidogrel oxalate according to claim 9, characterized in that equimolar amounts of oxalic acid are reacted with Clopidogrel base in a suitable solvent, preferably in an alcohol, ether, a nitrile, and/or an aqueous solvent mixtures thereof, preferably in isopropanol and/or diisopropylether and aqueous solvent mixtures, preferably thereof, with a water content of preferably less than 10% by weight of water (<10% by weight), whereby the compound is isolated by solvent abstraction.

13. Method of making Clopidogrel napsylate Form A according to claim 2, characterized in that equimolar amounts of naphthalene-2-sulfonic acid are combined with Clopidogrel base in a suitable solvent and initiating crystallization by inoculating the crystallization solution with Clopidogrel napsylate Form A, preferably in primary and/or secondary alcohols, ethers, nitrites, toluene and aqueous solvent mixtures, preferably thereof, with a water content of preferably less than 10% by weight (<10% by weight), preferably at a temperature working range between 20° C. and 60° C., preferably in isopropanol-water mixtures, diisopropylether, preferred is isopropanol-water mixtures.

14. Method of making Clopidogrel napsylate Form A according to claim 2, characterized in that said Clopidogrel napsylate Form A is made from another Clopidogrel salt by salt transformation in the presence of naphthalene-2-sulfonic acid salts, preferably sodium-2-naphthylsulfonate, preferably from Clopidogrel hydrobromide, preferably in isopropanol, diisopropylether, and/or aqueous solvent mixtures, preferably thereof, with a water content of preferably less than 10% by weight of water preferably at a working temperature range of 20° C. to 60° C.

15. Method of making Clopidogrel napsylate Form A according to claim 2, characterized in that said Clopidogrel napsylate Form A is obtained directly and without inoculation, by reacting equimolar amounts of naphthalene-2-sulfonic acid with Clopidogrel base in a suitable solvent, preferably in isopropanol, diisopropylether, and/or aqueous solvent mixtures, preferably thereof, with a water content of preferably less than 10% by weight wherein said naphthalene-2-sulfonic acid has a purity of at least 99.5% by weight and preferably, wherein the content of naphthalene-1-sulfonic acid is less than 0.5% by weight.

16. Method of making Clopidogrel napsylate Form B according to claim 2, characterized in that equimolar amounts of naphthalene-2-sulfonic acid are dissolved with Clopidogrel base in a suitable solvent and crystallization is initiated by inoculation with Clopidogrel napsylate Form B, preferably in a primary and/or secondary alcohol, a nitrile, toluene and/or an aqueous solvent mixture, preferably thereof, with a water content of preferably less than 10% by weight of water, preferably in isopropanol as a solvent, preferably in a strongly over saturated crystallizing solution (>20%), at a temperature working range from 15° C. to 20° C.

17. Method of making Clopidogrel napsylate Form B according to claim 2, characterized in that said Clopidogrel napsylate Form B is obtained by salt transformation from another Clopidogrel salt, preferably Clopidogrel hydrobromide, in the presence of a naphthalene-2-sulfonic acid salt, preferably sodium-2-naphthylsulfonate, or by recrystallization from Clopidogrel napsylate Form A, by inoculating the solution with the Clopidogrel napsylate Form B, preferably in isopropanol and/or diisopropylether, and aqueous solvent mixtures, preferably thereof, with a water content of preferably less than 10% by weight (<10% by weight) of water, preferably at a temperature working range of 15° C. to 20° C.

18. Method of making Clopidogrel napsylate Form B according to claim 2, characterized in that said Clopidogrel napsylate Form B is obtained directly without inoculation by reacting equimolar amounts of naphthalene-2-sulfonic acid with Clopidogrel base in a suitable solvent, preferably isopropanol and/or diisopropylether, and/or aqueous solvent mixtures, preferably thereof, with a water content of preferably less than 10% by weight of water, wherein the naphthalene-2-sulfonic acid used has a purity of less than 99.0% by weight and preferably if its content of naphthalene-1-sulfonic acid is higher than 1.0% by weight.

19. Pharmaceutically active compositions which contain at least one compound according to claim 1 in a pharmaceutically effective concentration.

20. Use of the compounds according to claim 1 for the preparation of pharmaceutically active compositions which contain at least one of said compounds in a pharmaceutically effective concentration.
